(19) United States
(12) Patent Application Publication
(10) Pub. No.: US 2026/0152477 A1
(43) Pub. Date: Jun. 4, 2026

(54) TOCOPHEROL DERIVATIVE, PREPARATION METHOD THEREFOR, AND USE THEREOF

(71) Applicant: Shanghai Coachchem Technology Co., Ltd., Shanghai (CN)

(72) Inventors: JIANG WU, Shanghai (CN); WEI ZHANG, Shanghai (CN); ANG GAO, Shanghai (CN); TENGFEI HAN, Shanghai (CN); BAOPING ZHOU, Shanghai (CN); DIE ZHANG, Shanghai (CN)

(73) Assignee: Shanghai Coachchem Technology Co., Ltd., Shanghai (CN)

(21) Appl. No.: 19/358,428

(22) Filed: Oct. 15, 2025

(30) Foreign Application Priority Data
Dec. 4, 2024 (CN) 202411770621.8

(51) Int. Cl. C07D 311/72; A61K 8/67; A61K 31/355; A61P 39/06; A61Q 19/00; B01J 27/18; B01J 31/02

(52) U.S. Cl. CPC C07D 311/72; A61K 8/678; A61K 31/355; A61P 39/06; A61Q 19/00; B01J 27/1806; B01J 31/0241; A61K 2800/522; B01J 2231/49

(57) ABSTRACT

The invention provides a tocopherol derivative, preparation method therefor, and uses thereof. The tocopherol derivative is a compound with the following structure.

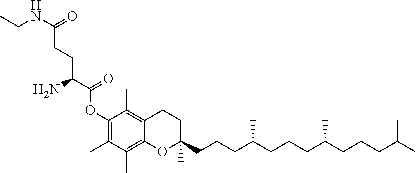

wherein R′ is hydrogen or an acetyl group; the theanine and tocopherol are esterified in the presence of a catalyst and a co-catalyst to obtain the tocopherol derivative. The compound can be used as an antioxidant in cosmetics, food and health products, pet products, etc. The method has mild reaction conditions, simple operation, high yield, high synthesis efficiency, economic and environmental protection, and has good application prospects.

### CROSS-REFERENCE TO RELATED APPLICATION

This application claims the priority benefit of China application serial no. 202411770621.8, filed on Dec. 4, 2024. The entirety of the above-mentioned patent application is hereby incorporated by reference herein and made a part of this specification.

### TECHNICAL FIELD

The present invention relates to a novel chemical compound and a method of preparation thereof, and more specifically, to a tocopherol derivative, its catalytic synthesis method, and use thereof.

### BACKGROUND

Tocopherol and theanine have attracted considerable attention in the industry due to their antioxidant functions. It is expected that combining the two may yield a product with synergistic or enhanced effects.

However, according to existing related technologies concerning the reaction conditions of phenols and acids, attempts have been made to condense theanine and tocopherol in tetrahydrofuran to obtain a synthetic product. During the reaction process, it was found that the scheme required harsh reaction conditions, and the yield and purity were unsatisfactory.

In another existing related technology, the acyl chloride derivative of theanine was condensed with tocopherol to obtain the desired synthetic product. However, it was also found that the reaction conditions remained harsh, and the yield and purity were similarly poor.

Therefore, there is an urgent need to develop a synthetic product based on tocopherol and theanine, together with a synthesis method that is efficient, energy-saving, and capable of reducing safety hazards and toxic or harmful issues in the production process.

### SUMMARY

The present invention aims to provide a synthetic product of tocopherol and theanine, namely a tocopherol derivative, and to overcome the disadvantages of conventional phenol-acid reactions, including low economic efficiency, harsh reaction conditions, and complicated operations. The invention provides a novel preparation method for tocopherol derivatives. The method is characterized by mild reaction conditions, simple operation, high yield, high efficiency, cost-effectiveness, and environmental friendliness, with promising application prospects.

Specifically, the present invention provides a tocopherol derivative characterized by the following structure:

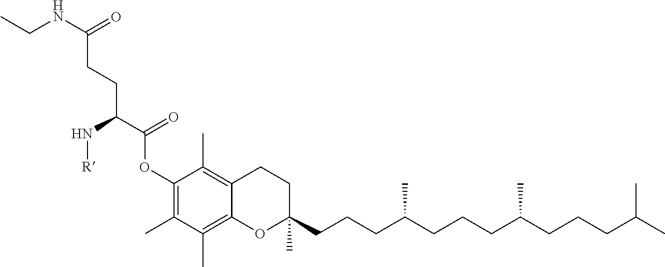

wherein R′ is hydrogen or an acetyl group.

Preferably, R′ is hydrogen. From the perspectives of product stability, usability, and diversification, one hydrogen atom of the NH₂ group may also be substituted with an acetyl group.

The tocopherol derivative can be used as an antioxidant and applied as an antioxidant additive in cosmetics, health foods, and pet products.

The present invention further provides a preparation method for the tocopherol derivative, characterized in that theanine and tocopherol are esterified in the presence of a catalyst and a co-catalyst to obtain the tocopherol derivative.

The catalyst is selected from compounds of the following structure:

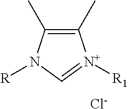

wherein R and R₁ are identical or different alkyl or aryl groups.

The specific reaction equation is shown as follows:

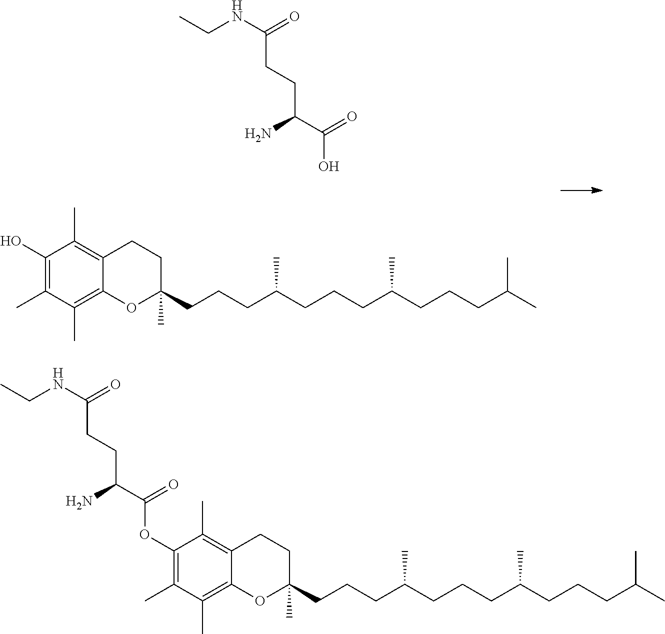

In the above catalyst, R and R₁ are preferably Me, n-Bu, or Ph. The most preferred catalysts are selected from the following compounds:

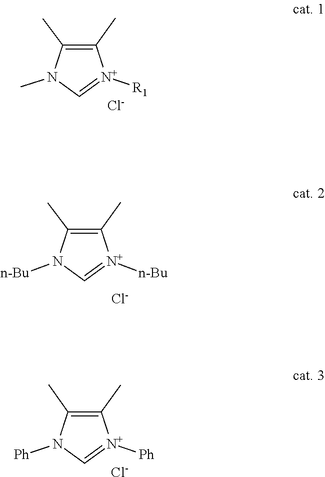

The reaction mechanism of the catalyst in the preparation of the tocopherol derivative is as follows: the acidic hydrogen of the cationic portion of the catalyst serves as a hydrogen bond donor to activate the carbonyl group of theanine, while the oxygen anion of the anionic portion of the catalyst acts as a hydrogen bond acceptor to activate the phenolic hydroxyl group of tocopherol, thereby promoting the esterification reaction.

Taking catalyst cat.1 as an example, the activation mechanism of the reaction can be described as follows:

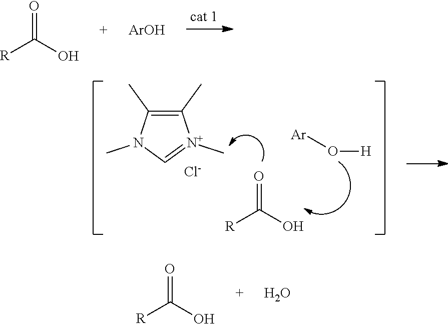

Furthermore, the preparation method of the tocopherol derivative according to the present invention is characterized in that: the molar amount of the catalyst added is 0.2-0.5% of the total molar amount of the reactants, preferably 0.2-0.4%, and most preferably 0.2-0.3%.

Further, the molar ratio of theanine to tocopherol is 1-1.5:1, preferably 1.2-1.5:1, and most preferably 1.4-1.5:1.

Further, the co-catalyst is sodium phosphate, which functions to activate the catalyst.

Further, the molar ratio of the co-catalyst to theanine is 0.1-0.5:1, preferably 0.1-0.3:1, and most preferably 0.1-0.2:1.

Further, the preparation method is characterized in that theanine, tocopherol, the catalyst, and the co-catalyst are mixed and reacted under stirring at room temperature for 4-6 hours, followed by post-treatment and purification to obtain the product.

Further, the reaction is carried out in a solvent. The solvent is selected from one or more of halogenated alkanes (e.g., dichloromethane, chloroform), ethers (e.g., tetrahydrofuran, dioxane, tert-butyl methyl ether, diethyl ether), nitriles (e.g., acetonitrile), amides (e.g., N,N-dimethylformamide), and sulfoxides (e.g., dimethyl sulfoxide).

Further, the method may additionally comprise adding molecular sieves in an amount of 0.1-1% of the total weight of the raw materials, preferably 0.1-0.5%, and most preferably 0.1-0.3%.

Further, the catalyst and the co-catalyst are used to catalyze the esterification reaction.

The present invention also proposes the use of the tocopherol derivative as an antioxidant.

### BRIEF DESCRIPTION OF THE DRAWINGS

FIG. 1. illustrates the hydrogen spectrum of the tocopherol derivative provided in Example 1.

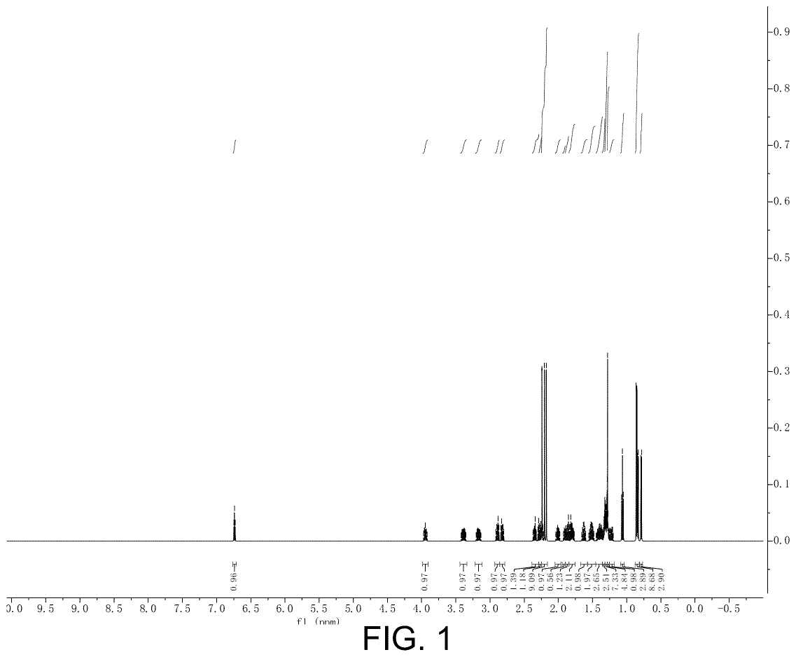

FIG. 2. illustrates the reference curve for the scavenging effect of ascorbic acid (VC) on DPPH radicals.

FIG. 3. illustrates the scavenging effect of the test product on DPPH radicals.

FIG. 4. illustrates the scavenging effect of the test product on ·OH.

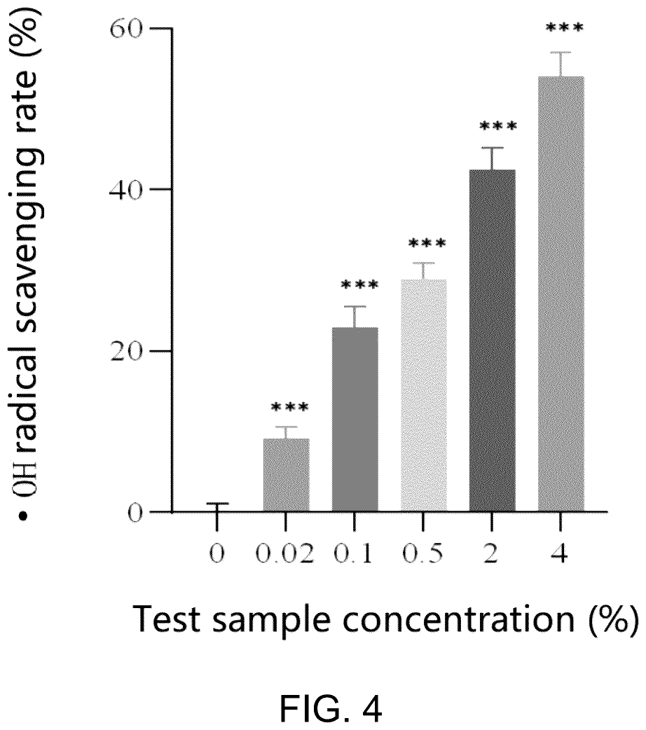

### DESCRIPTION OF THE EMBODIMENTS

Theanine, tocopherol, sodium phosphate, catalyst, and co-catalyst were mixed and stirred at room temperature for 4 hours. The reaction mixture was subjected to post-treatment and purification to afford the target product.

The structure of the product is represented as follows:

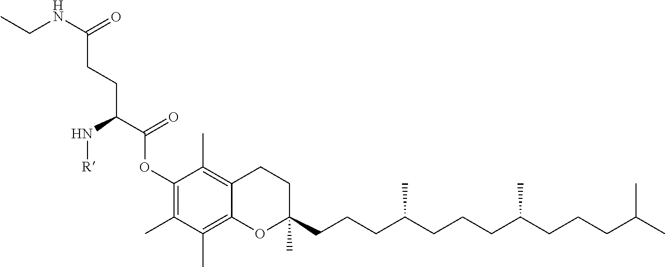

R′═H; based on the requirements of derivative products, the obtained product may further react with an acetylation reagent (e.g., acetyl chloride) to yield a derivative in which R′ is an acetyl group.

The catalyst is selected from

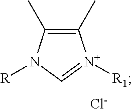

R and R₁ are identical or different alkyl or aryl groups. Examples of alkyl groups include straight-chain or branched alkyl groups containing no more than 10 carbon atoms, such as methyl, ethyl, propyl, isopropyl, butyl, isobutyl, pentyl, 2-methylbutyl, etc. Examples of aryl groups include phenyl, benzyl, p-tolyl, o-tolyl, m-tolyl, xylyl, p-methoxyphenyl, p-chlorophenyl, m-methoxyphenyl, m-chlorophenyl, o-methoxyphenyl, and o-chlorophenyl.

The preferred catalysts are selected from cat. 1, cat.2, and cat.3.

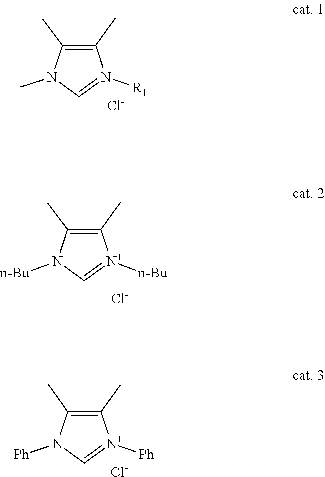

The co-catalyst is sodium phosphate. The reaction solvent is selected from halogenated hydrocarbons (e.g., dichloromethane, chloroform), ethers (e.g., tetrahydrofuran, dioxane, tert-butyl methyl ether, diethyl ether), nitriles (e.g., acetonitrile), amides (e.g., N,N-dimethylformamide), or sulfoxides (e.g., dimethyl sulfoxide), either alone or in combination.

The molar amount of the catalyst is 0.2-0.5% of the total molar amount of the reactants, or 2-10% of the total weight. The molar ratio of theanine to tocopherol is selected within 1-1.5:1. The molar ratio of sodium phosphate to theanine is selected within 0.1-0.5:1. Additionally, 0.1-1% by weight of molecular sieves relative to the total raw materials may be added.

Sources of Raw Materials:

| Reagent | Supplier | Specification | Notes |
|---|---|---|---|
| Theanine | Shandong Pingju | 1 kg, 99% | Configuration: L-theanine |
| | Biotechnology Co., Ltd. | | |
| Tocopherol | Macklin | 1 kg, 97% | Configuration: DL-α- |
| | | | tocopherol |
| Catalyst cat. 1 | Aladdin | 1 g, 99% | CAS: 141556-39-0 |
| Catalyst cat. 2 | Aladdin | 1 g, 99% | CAS: 1020098-95-6 |
| Catalyst cat. 3 | Aladdin | 1 g, 99% | CAS: 3052255-14-5 |
| Catalyst cat. 4 | Aladdin | 1 g, 99% | CAS: 54016-70-5 |
| Catalyst cat. 5 | Aladdin | 1 g, 99% | CAS: 2107416-41-9 |
| Sodium | Shanghai Haohong | 1 kg, 98% | |
| phosphate | Biomedical Technology Co., | | |
| | Ltd. | | |
| Tetrahydrofuran | Shanghai Haohong | AR | |
| | Biomedical Technology Co., | | |
| | Ltd. | | |
| Sodium | Shanghai Haohong | 1 kg, 98% | |
| bicarbonate | Biomedical Technology Co., | | |
| | Ltd. | | |
| Sodium | Shanghai Haohong | 1 kg, 98% | |
| chloride | Biomedical Technology Co., | | |
| | Ltd. | | |

### PREFERRED EMBODIMENTS

### Example 1. Preparation of Theanine Tocopherol Ester Catalyzed by Cat.1

Into a 250 mL three-neck flask were added theanine (5 g), tocopherol (3.9 g), cat. 1 (0.373 g), sodium phosphate (0.53 g), and tetrahydrofuran (100 mL). The mixture was stirred at room temperature for 4 h. The solid impurities were filtered off, 40 mL of water was added, and the mixture was stirred for 3 min. The organic phase was separated and dried to afford 5.9 g of pure theanine tocopherol ester (purity 97.6%), yield 88%. ¹H NMR (400 MHZ, CDCl₃) δ 6.77 (t, J=4.1 Hz, 1H), 3.92-3.81 (m, 1H), 3.41 (dd, J=7.3, 6.6 Hz, 1H), 3.26-3.16 (m, 2H), 2.78 (dd, J=7.8, 5.0 Hz, 1H), 2.72 (dd, J=7.8, 5.0 Hz, 1H), 2.38-2.25 (m, 2H), 2.19-2.04 (m, 7H), 2.04-1.90 (m, 1H), 1.75 (dd, J=7.8, 5.0 Hz, 1H), 1.73-1.19 (m, 25H), 1.13 (t, J=6.4 Hz, 3H), 0.85 (dd, J=6.8, 4.3 Hz, 9H), 0.79 (d, J=6.7 Hz, 3H). LC-MS: 587.75 [M+H]⁺.

### Example 2: Preparation of Tocopherol-Theanine Ester Catalyzed by Cat.2

Into a 250 mL three-neck flask were added theanine (5 g), tocopherol (3.9 g), cat.2 (0.363 g), sodium phosphate (0.53 g), and tetrahydrofuran (100 mL) as solvent. The mixture was stirred at room temperature for 4 hours. The solid impurities were filtered off, followed by addition of 40 mL of water, stirring for 3 minutes, separation of the organic phase, and drying to obtain pure tocopherol-theanine ester, 5.2 g (purity 94.3%), with a yield of 75%.

### Example 3: Preparation of Tocopherol-Theanine Ester Catalyzed by Cat.3

Into a 250 mL three-neck flask were added theanine (5 g), tocopherol (3.9 g), cat.3 (0.353 g), sodium phosphate (0.53 g), and tetrahydrofuran (100 mL) as solvent. The mixture was stirred at room temperature for 4 hours. The solid impurities were filtered off, followed by addition of 40 mL of water, stirring for 3 minutes, separation of the organic phase, and drying to obtain pure tocopherol-theanine ester, 6.2 g (purity 99.1%), with a yield of 92%.

### Example 4: Preparation of Tocopherol-Theanine Ester Catalyzed by Cat.3

Into a 250 mL three-neck flask were added theanine (5 g), tocopherol (3.9 g), cat.3 (0.353 g), sodium phosphate (0.53 g), and tetrahydrofuran (100 mL) as solvent. The mixture was stirred at room temperature for 4 hours. The solid impurities were filtered off, followed by addition of 40 mL of saturated sodium bicarbonate solution, stirring for 3 minutes, separation of the organic phase, and drying to obtain pure tocopherol-theanine ester, 6.4 g (purity 99.3%), with a yield of 94%.

### Example 5: Preparation of Tocopherol-Theanine Ester Catalyzed by Cat.3 (Optimum Conditions)

Into a 250 mL three-neck flask were added theanine (5 g), tocopherol (3.9 g), cat.3 (0.353 g), sodium phosphate (0.53 g), molecular sieves (0.2 g), and tetrahydrofuran (100 mL) as solvent. The mixture was stirred at room temperature for 4 hours. The solid impurities were filtered off, followed by addition of 40 mL of saturated sodium bicarbonate solution, stirring for 3 minutes, separation of the organic phase, then addition of 40 mL of saturated sodium chloride solution, and drying to obtain pure tocopherol-theanine ester, 6.9 g (purity 99.7%), with a yield of 98%.

### Comparative Example 1

Into a 250 mL three-neck flask were added theanine (5 g), tocopherol (3.9 g), and tetrahydrofuran (100 mL) as solvent. The mixture was stirred at room temperature for 4 hours. The solid impurities were filtered off, followed by addition of 40 mL of saturated sodium bicarbonate solution, stirring for 3 minutes, separation of the organic phase. Chromatographic analysis showed almost no product.

### Comparative Example 2

Into a 250 mL three-neck flask were added theanine (5 g), tocopherol (3.9 g), cat.3 (0.35 g), and tetrahydrofuran (100 mL) as solvent. The mixture was stirred at room temperature for 4 hours. The solid impurities were filtered off. Chromatographic analysis showed almost no product.

Comparative Example 3 Into a 250 mL three-neck flask were added theanine (5 g), tocopherol (3.9 g), cat.4

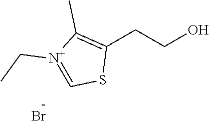

(0.35 g), and tetrahydrofuran (100 mL) as solvent. The mixture was stirred at room temperature for 4 hours. The solid impurities were filtered off, followed by addition of 40 mL of saturated sodium bicarbonate solution, stirring for 3 minutes, separation of the organic phase. Chromatographic analysis showed almost no product.

### Comparative Example 4

Into a 250 mL three-neck flask were added theanine (5 g), tocopherol (3.9 g), cat.5

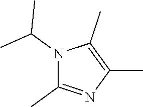

(0.35 g), and tetrahydrofuran (100 mL) as solvent. The mixture was stirred at room temperature for 4 hours. The solid impurities were filtered off, followed by addition of 40 mL of saturated sodium bicarbonate solution, stirring for 3 minutes, separation of the organic phase. Chromatographic analysis showed almost no product.

Comparative Example 5 Into a 250 mL three-neck flask were added theanine (5 g), tocopherol (3.9 g), boric acid (0.3 g), oxalic acid (0.3 g), and tetrahydrofuran (100 mL) as solvent. The mixture was stirred at room temperature for 4 hours. The solid impurities were filtered off, followed by addition of 40 mL of saturated sodium bicarbonate solution, stirring for 3 minutes, separation of the organic phase. Chromatographic analysis showed almost no product.

### Experimental Example 6

Into a 250 mL three-neck flask were added theanine (5 g), tocopherol (3.9 g), sodium phosphate (0.1 g), cat.3 (0.35 g), and tetrahydrofuran (100 mL) as solvent. The mixture was stirred at room temperature for 4 hours. The solid impurities were filtered off, followed by addition of 40 mL of water, stirring for 3 minutes, separation of the organic phase, and drying to obtain pure tocopherol-theanine ester, 4.1 g (purity 98.4%).

### Experimental Example 7

Into a 250 mL three-neck flask were added theanine (5 g), tocopherol (3.9 g), sodium phosphate (0.9 g), cat.3 (0.35 g), and tetrahydrofuran (100 mL) as solvent. The mixture was stirred at room temperature for 4 hours. The solid impurities were filtered off, followed by addition of 40 mL of water, stirring for 3 minutes, separation of the organic phase, and drying to obtain pure tocopherol-theanine ester, 4.3 g (purity 98.1%).

### Experimental Example 8

Into a 250 mL three-neck flask were added theanine (5 g), tocopherol (3.9 g), sodium phosphate (0.5 g), cat.3 (0.35 g), and ethanol (100 mL) as solvent. The mixture was stirred at room temperature for 4 hours. The solid impurities were filtered off, followed by addition of 40 mL of water, stirring for 3 minutes, separation of the organic phase, and drying to obtain pure tocopherol-theanine ester, 2.8 g (purity 91.1%).

### Experimental Example 9

Into a 250 mL three-neck flask were added theanine (5 g), tocopherol (3.9 g), PBS (0.5 g), cat.3 (0.35 g), and tetrahydrofuran (100 mL) as solvent. The mixture was stirred at room temperature for 4 hours. The solid impurities were filtered off, followed by addition of 40 mL of water, stirring for 3 minutes, separation of the organic phase, and drying to obtain pure tocopherol-theanine ester, 3.3 g (purity 89.6%).

### Experimental Example 10 Antioxidant Effect Test of Tocopherol-Theanine Ester (the Test Sample being the Purified Tocopherol-Theanine Ester Synthesized in Example 1)

### 1. Determination of DPPH Radical Scavenging Capacity

### 1.1 Experimental Principle

The DPPH radical scavenging activity assay is an in vitro method to evaluate antioxidant activity. DPPH is a stable macromolecular free radical in organic solvents and exhibits a purple color in methanol or ethanol with maximum absorbance at 517 nm. The DPPH colorimetric method is based on the principle that a radical scavenger can donate an electron to pair with the unpaired electron of DPPH, thereby changing the purple color at 517 nm to yellow. The degree of absorbance change is linearly correlated with the extent of free radical scavenging, i.e., the stronger the scavenging ability of the radical scavenger, the lower the absorbance.

1.2 Experimental ReagentsDPPH (Sigma), PBS (Gibco), absolute ethanol (Sinopharm Reagent), petroleum ether (Sinopharm Reagent), vitamin C (CNW), absolute ethanol (Sinopharm Reagent). Main equipment: microplate reader (Tecan, Spark), micro-shaker (Qilinbeier, TS-92).

### 1.3 Experimental Methods

### 1.3.1 Construction of Reference Curve for DPPH Radical Scavenging by System Reference Substance

Ascorbic acid (VC) was used as the system reference substance and diluted with PBS to 12.5, 25, 50, 100, and 200 μg/mL at five gradient concentrations. The assay was conducted according to method 1.3.2, and results were calculated. A reference curve was plotted with the reference substance concentration as the x-axis and the DPPH radical scavenging rate as the y-axis.

### 1.3.2 In Vitro DPPH Radical Scavenging Assay

The test sample was prepared into solutions of corresponding concentrations. Reaction systems were formulated according to Table 1 with the specified reagent additions, mixed thoroughly, with five replicate wells for each concentration and one background control well.

TABLE 1
Reaction System of DPPH Radical Scavenging Assay

| Unit (μL) | 1 | 2 | 1 | 2 |
|---|---|---|---|---|
| DPPH ethanol solution | 180 | 10 | 180 | 0 |
| Test solution | 0 | 0 | 20 | 20 |
| Absolute ethanol | 0 | 180 | 0 | 180 |
| PBS | 20 | 20 | 0 | 0 |

The reaction systems were incubated at room temperature in the dark for 30 minutes. At the end of the reaction, absorbance (OD) values were measured at 517 nm, and the DPPH radical scavenging rate of the test sample was calculated using the following formula:

Scavenging rate of test sample on DPPH radicals=[(C1−C2)−(T1−T2)]/(C1−C2)×100%
Wherein: C1—absorbance of blank system containing DPPH
C2—absorbance of blank system without DPPH
T1—absorbance of test sample group containing DPPH
T2—absorbance of test sample group without DPPH

### 1.4 Results of In Vitro DPPH Radical Scavenging Assay

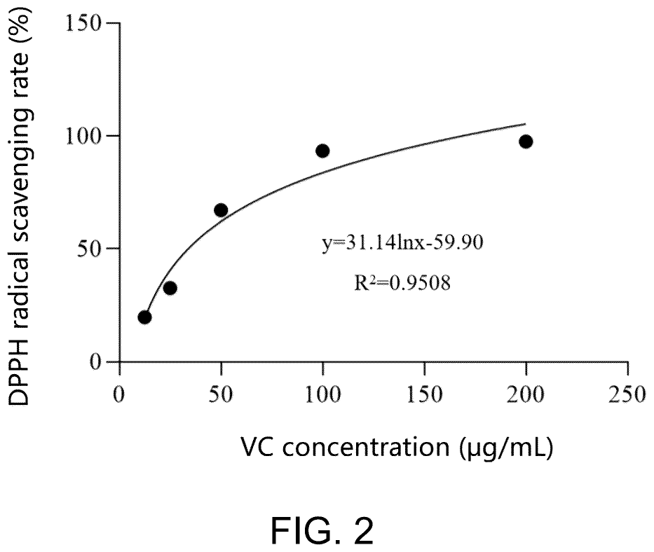

1.4.1 System Reference Curve of DPPH Radical Scavenging. See Table 2 and FIG. 2.

TABLE 2
Analysis of System Reference Substance Results

VC concentration (μg/mL)

| | 200 | 100 | 50 | 25 | 12.5 |
|---|---|---|---|---|---|
| DPPH radical | 99.60 | 90.41 | 67.87 | 30.55 | 20.71 |
| scavenging | 99.65 | 90.43 | 72.65 | 33.04 | 21.33 |
| rate (%) | 96.14 | 91.70 | 65.65 | 31.49 | 16.32 |
| | 100.61 | 96.01 | 64.59 | 34.01 | 22.89 |
| | 91.05 | 97.86 | 64.51 | 33.93 | 17.46 |

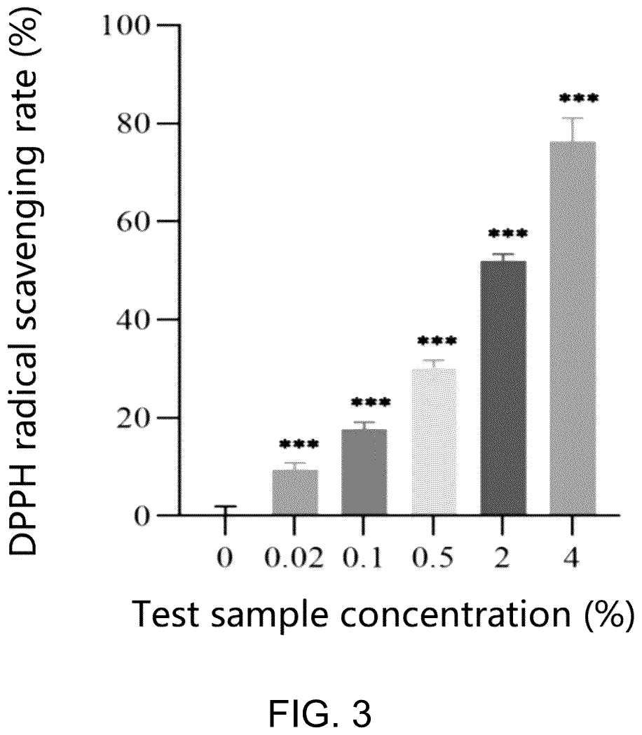

1.4.2 In Vitro DPPH Radical Scavenging Results of the Test Sample. See Table 3 and FIG. 3.

TABLE 3
Analysis of DPPH Radical Scavenging Results of Test Sample

| Test sample | DPPH radical | | |
|---|---|---|---|
| concentration | scavenging rate | | |
| (% v/v) | (% v/v) | p | Significance |
| 0 | 0.31 ± 1.65 | / | / |
| 0.02 | 9.52 ± 1.34 | <0.001 | *** |
| 0.1 | 17.71 ± 1.40 | <0.001 | *** |
| 0.5 | 30.36 ± 1.44 | <0.001 | *** |
| 2 | 52.02 ± 1.32 | <0.001 | *** |
| 4 | 76.40 ± 4.73 | <0.001 | *** |

Note:
The number of asterisks indicates the level of statistical significance, where
*** denotes high significance and
* denotes low significance.

### 1.5 Conclusion

At concentrations of 0.02%-4%, the test sample significantly increased the DPPH radical scavenging rate compared with the control group (p<0.001), demonstrating antioxidant activity.

### 2. Determination of Hydroxyl Radical (OH) Scavenging Capacity

### 2.1 Experimental Principle

When the antioxidant function of the body declines or cell damage occurs, excessive free radicals are produced. Among reactive oxygen species, hydroxyl radical (OH) has the highest reactivity (10⁷−10¹⁰ M⁻¹ S⁻¹) and is the most harmful to the body. Hydroxyl radicals are generated through the Fenton reaction: H₂O₂+Fe²⁺→·OH+H₂O+Fe³⁺. The generated ·OH reacts with salicylic acid to produce a compound with specific absorbance at 520 nm. The addition of a test sample capable of scavenging ·OH reduces the amount of ·OH generated, thereby decreasing the amount of colored compound formed. The degree of absorbance change is related to the extent of ·OH scavenging; i.e., the stronger the scavenging ability, the lower the absorbance.

### 2.2 Experimental Methods

The test sample was prepared into solutions of corresponding concentrations. The reagents were added according to the reaction system in Table 4, and absorbance values were measured at 520 nm. The scavenging rate was calculated according to the following formula. The test sample was tested at five gradient concentrations, with each concentration measured in five replicates.

Scavenging rate of test sample on ·OH radicals=[(A−C)−(B−D)]/(A−C)×100%

TABLE 4
Reaction System of Hydroxyl Radical Scavenging Assay

| A (Control) | B (Test sample) | C (Control reference) | C (Sample reference) |
|---|---|---|---|
| 0.2 mL distilled water | 0.2 mL test solution | 0.2 mL distilled water | 0.2 mL test solution |
| 0.2 mL FeSO₄ | 0.2 mL FeSO₄ | 0.2 mLFeSO₄ | 0.2 mL FeSO₄ |
| 0.4 mL H₂O₂ | 0.4 mL H₂O₂ | 0.4 mL H₂O₂ | 0.4 mL H₂O₂ |
| Reaction 10 min | Reaction 10 min | Reaction 10 min | Reaction 10 min |
| 0.2 mL salicylic acid | 0.2 mL salicylic acid | 0.2 mL absolute | 0.2 mL absolute |
| ethanol solution | ethanol solution | ethanol solution | ethanol solution |

### 2.3 Results of In Vitro Hydroxyl Radical Scavenging Test See Table 5.

TABLE 5
Analysis of Hydroxyl Radical Scavenging Results of Test Sample

| Test sample | | | |
|---|---|---|---|
| concentration | •OH scavenging | | |
| (% v/v) | rate (% v/v) | p | Significance |
| 0 | 0.11 ± 1.65 | / | / |
| 0.02 | 9.30 ± 1.34 | <0.001 | *** |
| 0.1 | 22.98 ± 1.40 | <0.001 | *** |
| 0.5 | 29.00 ± 1.44 | <0.001 | *** |
| 2 | 42.50 ± 1.32 | <0.001 | *** |
| 4 | 54.10 ± 4.73 | <0.001 | *** |

Note:
The number of asterisks indicates the level of statistical significance, where
*** denotes high significance and
* denotes low significance.

### 2.4 Experimental Conclusion

At concentrations of 0.02%-4%, the test sample significantly increased hydroxyl radical scavenging rates compared with the control group (p<0.001), demonstrating antioxidant activity.

What is claimed is:

1. A tocopherol derivative, wherein the tocopherol derivative is a compound shown in following structure: 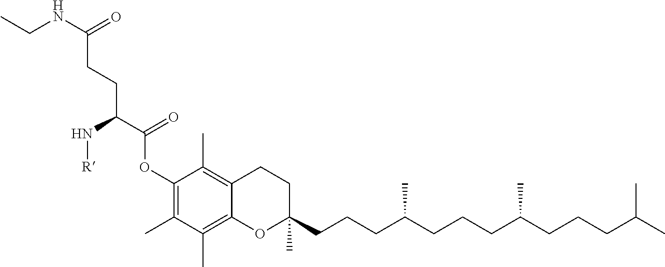
wherein R′ is hydrogen or an acetyl group.

2. A method for preparing a tocopherol derivative of claim 1, wherein a theanine and a tocopherol are esterified in presence of a catalyst and a co-catalyst to obtain the tocopherol derivative;
the catalyst is selected from compounds shown in following structure: 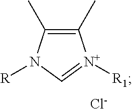
R and R₁ are identical or different alkyl or aryl groups;
the alkyl groups include straight-chained or branched alkyl groups containing no more than 10 carbon atoms; the aryl groups selected from phenyl, benzyl, p-tolyl, o-tolyl, m-tolyl, xylyl, p-methoxyphenyl, p-chlorophenyl, m-methoxyphenyl, m-chlorophenyl, o-methoxyphenyl, and o-chlorophenyl.

3. The method of claim 2, wherein a molar amount of the catalyst added is 0.2-0.5% of a total molar amount of reactants.

4. The method of claim 2, wherein a molar ratio of the theanine to the tocopherol is 1-1.5:1.

5. The method of claim 2, wherein the co-catalyst is sodium phosphate.

6. The method of claim 2, wherein a molar ratio of the co-catalyst to the theanine is 0.1-0.5:1.

7. The method of claim 2 wherein, the theanine, the tocopherol, the catalyst, and the co-catalyst are mixed and reacted under stirring at room temperature for 4-6 hours, followed by post-treatment and purification to obtain products.

8. The method of claim 7, wherein molecular sieves are added.

9. Use of the tocopherol derivative of claim 1 as an antioxidant.

10. Use of the tocopherol derivative prepared by the method of claim 2 as an antioxidant.
